(19) United States
(12) Patent Application Publication
(10) Pub. No.: US 2005/0064045 A1
(43) Pub. Date: Mar. 24, 2005

(54) Injectable therapeutic formulations

(76) Inventors: Sheng-Ping Zhong, Shrewsbury, MA (US); Wendy Naimark, Cambridge, MA (US); Arthur Madenjian, Winchester, MA (US); Weenna Bucay-Couto, Burlington, MA (US); Enxin Ma, Natick, MA (US)

Correspondence Address: MAYER, FORTKORT & WILLIAMS, PC, 251 NORTH AVENUE WEST, 2ND FLOOR, WESTFIELD, NJ, 07090, US

(21) Appl. No.: 10/667,151

(22) Filed: Sep. 18, 2003

(51) Int. Cl. A61K 33/14; A61K 31/045

(52) U.S. Cl. 424/680; 514/724

(57) ABSTRACT

Sterile injectable formulations, which comprise the following: (a) a ablation agent in an amount effective to cause necrosis of tissue, and (b) a biodisintegrable viscosity adjusting agent in an amount effective to render the formulation highly viscous, (c) an optional imaging contrast agent, (d) an optional therapeutic agent, and (e) an optional liquid selected from water and an organic solvent. Also described are novel prostatic ablation formulations, which comprise a prostatic ablation agent selected from free-radical generating ablation agents, oxidizing ablation agents and tissue fixing ablation agents. Further aspects of the invention relate to methods of treating a variety of diseases and conditions, including benign prostatic hypertrophy, in which above injectable formulations are injected into the tissue of a subject, optionally with the assistance of a non-invasive imaging technique.

### FIELD OF THE INVENTION

The present invention relates to formulations and methods for chemoablation of tissue, such as prostate tissue. More particularly, the present invention relates to high-viscosity formulations for direct injection into tissue (e.g., the prostate), thereby leading to ablation of the tissue.

### BACKGROUND OF THE INVENTION

Prostate diseases such as prostatitis, benign prostatic hypertrophy, prostatodynia, and prostate carcinoma afflict many adult males. The largest population of men stricken with prostate problems is men over age fifty, although inherited prostate problems can appear in much younger men.

Benign prostatic hypertrophy is a condition where the prostate over-grows or becomes enlarged. Prostate growth is controlled by androgen receptors found in the prostate gland. When the androgen receptors are stimulated by 5-alpha-dihydrotesterone (DHT), they cause the prostate to grow. DHT is produced by an enzymatic conversion of testosterone in the prostate.

Over the past twenty years, a variety of approaches have been developed to treat benign prostatic hypertrophy. In general, these approaches alter the tissue volume or the biochemistry of the prostate, and they include the application of heat, cold, chemical agents, pharmaceutical agents and radiation. In recent years, a number of minimally invasive technologies have been developed, including radiation, RF ablation, microwave ablation, cryogenic ablation/freezing, and chemo-ablation.

Chemo-ablative approaches, including injection of alcohol or salt solutions, have been evaluated for the treatment of benign prostatic hypertrophy. However, the lack of delivery control when administering presently known liquids has led to unpredictable retention, leading to nonspecific ablation of both the prostate and surrounding tissues and organs.

### SUMMARY OF THE INVENTION

The above and other needs and challenges are addressed by the present invention.

In this regard, various aspects of the present invention concern sterile injectable formulations that comprise the following: (a) an ablation agent in an amount effective to cause necrosis of tissue upon injection, (b) a biodisintegrable viscosity adjusting agent in an amount effective to render the injectable formulation highly viscous, (c) an optional imaging contrast agent and (d) an optional additional therapeutic agent.

Other aspects of the present invention relate to methods of treatment in which injectable formulation like those above are injected into the tissue of a subject. Tissue benefiting from such treatment include prostate tissue, kidney tissue, liver tissue, bladder tissue, benign tumors and malignant tumors.

Other aspects of the present invention concern sterile injectable prostatic formulations which comprise the following: (a) a prostatic ablation agent in an amount effective to cause necrosis of prostate tissue, and (b) a biodisintegrable viscosity adjusting agent in an amount effective to render the prostatic formulation highly viscous, (c) an optional imaging contrast agent and (d) an optional additional therapeutic agent. Other aspects of the present invention are directed to injectable chemoablation formulations which comprise novel chemical agents for tissue ablation and, optionally, viscosity adjusting agents, contrast agents, additional therapeutic agents and their combinations.

Still other aspects of the present invention relate to methods of treating benign prostatic hypertrophy, prostatitis, and prostate cancer in which the injectable prostatic formulations of the present invention are injected into the prostate of a subject, optionally with the assistance of a non-invasive imaging technique.

An advantage of the present invention is that injectable formulations can be provided, which have improved retention of ablative agents in prostatic and other tissue, thereby improving delivery efficiency while minimizing adverse effects such as nonspecific damage.

Another advantage of the present invention is that injectable formulations can be provided, which are capable of being detected by noninvasive monitoring techniques, including ultrasound, x-ray fluoroscopy, and magnetic resonance imaging (MRI). In this way, the volume and location of the injectable formulations can be more precisely monitored and controlled.

Another advantage of the present invention is that injectable formulations can be provided, which display good retention in tissue such as prostate tissue, while at the same time being capable of being injected into tissue using conventional syringes, injection catheters, and so forth.

Another advantage of the present invention is that injectable formulations can be provided, which display controlled release of chemoablative and other therapeutic agents.

Yet another advantage of the present invention is that injectable formulations having novel chemoablative agents can be provided.

These and other embodiments and advantages of the present invention will become immediately apparent to those of ordinary skill in the art upon review of the Detailed Description and claims to follow.

### DETAILED DESCRIPTION OF THE INVENTION

According to an aspect of the present invention, chemoablative injection formulations are provided, which contain (a) at least one chemical ablation agent that is present in an amount effective to produce necrosis in tissue that is exposed to the formulation, and (b) at least one viscosity adjusting agent that is present in an amount effective to produce a high viscosity formulation.

“Highly viscous” and “high viscosity” are used herein to describe fluids having a kinematic viscosity greater than 1000 cps as measured on a Brookfield Kinematic Viscometer, model HBDV-II+CP with a CPE-40 cone spindle, set at 37° C. temperature, and using 0.5 rpm speed setting.

In general, ablation agents are materials whose inclusion in the injectable formulations of the present invention in sufficient amounts will result in necrosis (death) of tissue, such as prostatic tissue, upon injection of the formulation into the tissue.

In some embodiments, the ablation agents are osmotic-stress-generating agents, for example, a salt, such as sodium chloride or potassium chloride. The process of osmosis is the passage of at least one diffusible species (commonly, water) through a semipermeable membrane (e.g., the membranes that surround all cells in the body), which membrane simultaneously prevents the passage of at least one non-diffusible species (e.g., salt in salt water). In osmosis, the passage of the diffusible species is from a less concentrated solution (with respect to the non-diffusible species) through the membrane to a more concentrated one. What determines the relative concentration of the diffusible species is the amount of non-diffusible species present on either side of the membrane. Osmotic pressure is generated whenever environments of different water concentration are separated by a semipermeable membrane, and will remain until the two solutions are of equal concentration. This is why cells frequently swell (and even burst, in some cases), when placed in distilled water, and why they frequently shrivel when placed in aqueous solutions containing high concentrations of a non-diffusible agent, such as salt (or when exposed to pure salt). If cells are subjected to sufficient osmotic stress, they can dehydrate and die.

In other embodiments, the ablation agents are organic compounds that are toxic in high concentrations, while being non-toxic at lower concentrations, for example, ethanol. It is noted that alcohols, such as ethanol, like salt, can also dehydrate cells and tissues causing them to shrink and die.

In other embodiments, the ablation agents are free-radical generating agents, for example, hydrogen peroxide, potassium peroxide or other agents that can form free radicals in tissue, such as prostate tissue. Upon formation, the free radicals will attack the tissue to create necrosis. For example, free radicals can be formed by decomposition of the free-radical generating agent upon exposure to water, exposure to heat, exposure to light and/or exposure to exposure to other agents.

In other embodiments, the ablation agents are basic agents such as sodium hydroxide, acidic agents such as acetic acid and formic acid, and/or enzymes such as collagenase, hyaluronidase, pronase, and papain.

In still other embodiments, oxidizing agents, such as sodium hypochlorite, hydrogen peroxide or potassium peroxide, tissue fixing agents, such as formaldehyde, acetaldehyde or glutaraldehyde, or naturally occurring coagulants, such as gengpin, are used as ablation agents.

The amount of ablation agent will vary widely, with the amounts employed varying depending on the characteristics of the ablation agent, the tissue, and the biodisintegrable viscosity adjusting agent, among other factors. For example, where ethanol is selected as a prostatic ablation agent, the ratio of water:ethanol typically ranges from about 0:100 to 60:40, more typically from about 0:100 to 10:90. As another example, where salt (i.e., sodium chloride) is selected as a prostatic ablation agent, the concentration of salt in the formulation typically ranges from about 5 wt % to 35 wt % of the formulation.

As noted above, the injectable formulations of the present invention also comprise a viscosity adjusting agent in an amount effective to render the formulation highly viscous, for example, having a kinematic viscosity between about 5,000 and 200,000 cps, more typically between about 10,000 and 100,000 cps, and even more typically between about 20,000 and 40,000 cps.

By providing formulations having viscosities within these ranges, the formulations remain capable of being injected into tissue, such as prostatic tissue, using conventional injection equipment (e.g., syringes). However, due to their elevated viscosities, the formulations have improved retention within the tissue at the injection site, thereby improving the delivery efficiency of the ablation agents, while at the same time minimizing their adverse effects at locations removed from the injection site (e.g., nonspecific tissue damage).

The concentration of the viscosity adjusting agent that is used to provide the desired viscosity can vary widely. Commonly, the concentration of the viscosity adjusting agent is between about 1 and 20 wt %.

In many embodiments, the viscosity adjusting agents are biodisintegrable. A “biodisintegrable” viscosity adjusting agent is one that, once injected into tissue, such as the prostate, undergoes dissolution, degradation, resorption and/or other disintegration processes.

In many embodiments of the present invention, the viscosity adjusting agents are polymers, typically biocompatible, water soluble and/or hydrophilic polymers, which may be of natural or synthetic origin, and which may be homopolymers, copolymers or polymer blends. As the term is used herein, a “polymer” can consist of as few as two monomeric units, but will typically have many more. In some embodiments, for instance where an organic solvent such as dimethylsulfoxide (DMSO) is utilized, the viscosity adjusting agent can be relatively hydrophobic.

Examples of viscosity adjusting agents for the practice of the present invention include the following: cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, heparin, heparin sulfate, dermatan sulfate, proteins such as gelatin, collagen, albumin, and fibrin, other polymers, for example, carboxyvinyl polymers and their salts (e.g., carbomer), polyvinylpyrrolidone (PVP), polyacrylic acid and its salts, polyacrylamide, polyacilic acid/acrylamide copolymer, polyalkylene oxides such as polyethylene oxide, polypropylene oxide and poly(ethylene oxide-propylene oxide) (e.g., Pluronic acid from BASF), polyoxyethylene (polyethylene glycol), polyethyleneamine and polypyrridine, poly-metaphosphate (Kurrol salts), polyvinyl alcohol, additional salts and copolymers beyond those specifically set forth above, and blends of the foregoing (including mixtures of polymers containing the same monomers, but having different molecular weights), and so forth.

In some embodiments, the formulations of the present invention may be crosslinked, either ex vivo or in vivo. Beneficially, a crosslinking agent is injected into tissue either before or after the injection of the injectable formulation of the present invention.

Crosslinking agents for this purpose include ionic and covalent crosslinking agents. For example, polymers can be included within the formulations of the present invention, which can be ionically crosslinked with, for instance, polyvalent metal ions. Appropriate crosslinking ions include polyvalent cations selected from the group consisting of calcium, magnesium, barium, strontium, boron, beryllium, aluminum, iron, copper, cobalt, lead and silver cations ions. Polyvalent anions include phosphate, citrate, borate, succinate, maleate, adipate and oxalate anions. More broadly, crosslinking anions are commonly derived from polybasic organic or inorganic acids. Ionic crosslinking may be carried out by methods known in the art, for example, by contacting ionically crosslinkable polymers with an aqueous solution containing dissolved ions.

Polymers may also be included which can be covalently crosslinked using, for example, a polyfunctional crosslinking agent that is reactive with functional groups covalently bonded to the polymer structure. The polyfunctional crosslinking agent can be any compound having at least two functional groups that react with functional groups in the polymer. Various polymers described herein can be both covalently and ionically crosslinked.

Crosslinking is advantageous, for example, to improve fluid retention (e.g., by providing a more rigid material and/or by rendering the polymer less soluble in a particular environment).

The injection formulations of the present invention also optionally comprise therapeutic agents in addition to the ablation agents and viscosity adjusting agents described above. “Therapeutic agents”, “pharmaceutically active agents”, “pharmaceutically active materials”, “drugs” and other related terms may be used interchangeably herein and include genetic therapeutic agents, non-genetic therapeutic agents and cells. Therapeutic agents may be used singly or in combination. Therapeutic agents may be, for example, nonionic or they may be anionic and/or cationic in nature.

Exemplary non-genetic therapeutic agents for use in connection with the present invention include: (a) anti-thrombotic agents such as heparin, heparin derivatives, urokinase, and PPack (dextrophenylalanine proline arginine chloromethylketone); (b) anti-inflammatory agents such as dexamethasone, prednisolone, corticosterone, budesonide, estrogen, sulfasalazine and mesalamine; (c) anti-neoplastic/antiproliferative/anti-miotic agents such as paclitaxel, 5-fluorouracil, cisplatin, vinblastine, vincristine, epothilones, endostatin, angiostatin, angiopeptin, monoclonal antibodies capable of blocking smooth muscle cell proliferation, and thymidine kinase inhibitors; (d) anesthetic agents such as lidocaine, bupivacaine and ropivacaine; (e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promotors; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, aminoglycosides and nitrofurantoin; (m) cytotoxic agents, cytostatic agents and cell proliferation affectors; (n) vasodilating agents; (o) agents that interfere with endogenous vasoactive mechanisms; (p) inhibitors of leukocyte recruitment, such as monoclonal antibodies; (q) cytokines and (r) hormones.

Exemplary genetic therapeutic agents for use in connection with the present invention include anti-sense DNA and RNA as well as DNA coding for: (a) anti-sense RNA, (b) tRNA or rRNA to replace defective or deficient endogenous molecules, (c) angiogenic factors including growth factors such as acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor and insulin-like growth factor, (d) cell cycle inhibitors including CD inhibitors, and (e) thymidine kinase (“TK”) and other agents useful for interfering with cell proliferation. Also of interest is DNA encoding for the family of bone morphogenic proteins (“BMP's”), including BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNA's encoding them.

Vectors for delivery of genetic therapeutic agents include viral vectors such as adenoviruses, gutted adenoviruses, adeno-associated virus, retroviruses, alpha virus (Semliki Forest, Sindbis, etc.), lentiviruses, herpes simplex virus, replication competent viruses (e.g., ONYX-015) and hybrid vectors; and non-viral vectors such as artificial chromosomes and mini-chromosomes, plasmid DNA vectors (e.g., pCOR), cationic polymers (e.g., polyethyleneimine, polyethyleneimine (PEI)), graft copolymers (e.g., polyether-PEI and polyethylene oxide-PEI), neutral polymers PVP, SP1017.

(SUPRATEK), lipids such as cationic lipids, liposomes, lipoplexes, nanoparticles, or microparticles, with and without targeting sequences such as the protein transduction domain (PTD).

Cells for use in connection with the present invention include cells of human origin (autologous or allogeneic), including whole bone marrow, bone marrow derived mono-nuclear cells, progenitor cells (e.g., endothelial progenitor cells), stem cells (e.g., mesenchymal, hematopoietic, neuronal), pluripotent stem cells, fibroblasts, myoblasts, satellite cells, pericytes, cardiomyocytes, skeletal myocytes or macrophage, or from an animal, bacterial or fungal source (xenogeneic), which can be genetically engineered, if desired, to deliver proteins of interest.

A wide range of therapeutic agent loadings can be used in conjunction with the injectable formulations of the present invention, with the effective amount of loading being readily determined by those of ordinary skill in the art and ultimately depending, for example, upon the condition to be treated, the nature of the therapeutic agent itself, the tissue into which the injectable formulation is introduced, other formulation components, and so forth.

The injection formulations of the present invention also optionally include one or more imaging contrast agents, in addition to the ablation agents, viscosity adjusting agents, and optional therapeutic agents discussed above.

The ability to non-invasively image the body regions into which the formulations of the present invention have been introduced (and hence, by default, where they have not been introduced) is a valuable diagnostic tool. Among such currently available non-invasive imaging techniques are included magnetic resonance imaging (MRI), ultrasonic imaging, x-ray fluoroscopy, nuclear medicine, and others. Various imaging technologies have associated with them imaging contrast agents, i.e., substances that enhance the image produced by medical diagnostic equipment.

For example, x-ray based fluoroscopy is a diagnostic imaging technique that enables real-time patient monitoring of motion within a patient. To be fluoroscopically visible, formulations are typically rendered more absorptive of x-rays than the surrounding tissue. In various embodiments of the invention, this is accomplished by the use of contrast agents. Examples of contrast agents for use in connection with x-ray fluoroscopy include metals, metal salts and oxides (particularly bismuth salts and oxides), and iodinated compounds. Examples of such contrast agents include tungsten, platinum, tantalum, iridium, gold, or other dense metal, barium sulfate, bismuth subcarbonate, bismuth trioxide, bismuth oxychloride, metrizamide, iopamidol, iothalamate sodium, iodomide sodium, and meglumine.

Ultrasound and magnetic resonance imaging can provide two- and/or three-dimensional images of a portion of the body. Ultrasound and MRI are advantageous, inter alia, because they do not expose the subject or medical practitioner to harmful radiation and can provide detailed images of the observed area. These detailed images are valuable diagnostic aids to medical practitioners and can be used to more precisely control the quantity and location of the injection fluid of the present invention.

Magnetic resonance imaging (MRI) produces images by differentiating detectable magnetic species in the portion of the body being imaged. In the case of ¹H MRI, the detectable species are protons (hydrogen nuclei). In order to enhance the differentiation of detectable species in the area of interest from those in the surrounding environment, imaging contrast agents are often employed. These agents alter the magnetic environment of the detectable protons in the area of interest relative to that of protons in the surrounding environment and, thereby, allow for enhanced contrast and better images of the area of interest. For contrast-enhanced MRI, it is desirable that the contrast agent have a large magnetic moment, with a relatively long electronic relaxation time. Based upon these criteria, contrast agents such as Gd(III), Mn(II) and Fe(III) have been employed. Gadolinium(III) has the largest magnetic moment among these three and is, therefore, a widely-used paramagnetic species to enhance contrast in MRI. Chelates of paramagnetic ions such as Gd-DTPA (gadolinium ion chelated with the ligand diethylenetriaminepentaacetic acid) have been employed as MRI contrast agents. Chelation of the gadolinium or other paramagnetic ion is believed to reduce the toxicity of the paramagnetic metal by rendering it more biocompatible, and can assist in localizing the distribution of the contrast agent to the area of interest. Paramagnetic ion chelates can be, for example, attached to the viscosity adjusting agent or they can be simply admixed with the other components of the formulation. Further information can be found, for example, in U.S. Patent Application No. 20030100830 entitled “Implantable or insertable medical devices visible under magnetic resonance imaging,” the disclosure of which is incorporated herein by reference.

Ultrasound uses high frequency sound waves to create an image of living tissue. A sound signal is sent out, and the reflected ultrasonic energy, or “echoes,” used to create the image. Ultrasound imaging contrast agents are materials that enhance the image produced by ultrasound equipment. Ultrasonic imaging contrast agents introduced into the formulations of the present invention can be, for example, echogenic (i.e., materials that result in an increase in the reflected ultrasonic energy upon injection of the formulation) or echolucent (i.e., materials that result in a decrease in the reflected ultrasonic energy upon injection of the formulation).

Suitable ultrasonic imaging contrast agents for use in connection with the present invention include solid particles ranging from about 0.01 to 50 microns in largest dimension (e.g., the diameter, where spherical particles are utilized), more typically about 0.5 to 20 microns. Both inorganic and organic particles can be used. Examples include microparticles/microspheres of calcium carbonate, hydroxyapatite, silica, poly(lactic acid), and poly(glycolic acid). Microbubbles can also be used as ultrasonic imaging contrast agents as is known in the imaging art. The ultrasonic imaging contrast agents for use in connection with the present invention are preferably biocompatible and stable in the formulation. Concentrations of the ultrasonic imaging contrast agents typically range from 0.01 wt % to 10 wt % of the formulation, more typically 0.05 to 2 wt %, where solid particles are used.

Typically, the injection formulations of the present invention are formulated with water and/or an organic solvent. In some instances, the organic solvent(s) can also act as the ablation agent(s). Ethanol is a specific example. An example of an organic solvent which is not an effective ablation agent is DMSO.

Prior to injection, the dosage forms of the present invention are typically sterilized, for example, by exposing them to heat, radiation or ethylene oxide gas, or by preparing them under aseptic conditions.

Subjects for the procedures of the present invention include vertebrate subjects, typically mammalian subjects, more typically human subjects. The formulations of the present invention are injected into tissue of a subject by a variety of routes and using a variety of apparatuses.

Examples of tissue for treatment in accordance with the present invention include prostatic tissue, kidney tissue, liver tissue, bladder tissue, or any other organ or entity confined by a capsular membrane. The treated tissue may comprise benign tumor tissue or malignant tumor tissue. For example, disease states for which the treatment may be useful include, BPH, prostate cancer, prostititis, any other disease states occurring within a capsular membrane-confined organ. The solid salt dosage forms are inserted by any of a variety of routes, including transabdominal, transperineal, transcutaneous, transurethral, and transrectal routes of insertion. Other routes may be suitable depending on the application and location of tissue, which ensures access through the capsular membrane. Where prostatic tissue is to be treated, transperineal, transurethral, and transrectal routes are typically used, with transrectal administration being particularly beneficial.

For example, in some embodiments, the formulations of the present invention are injected into the prostate using conventional (or specially designed) syringes, injection catheters, and so forth. Typical forces that are required to push the fluid out of a 20 gauge needle, 6 inches long, using a 5 cc Becton Dickenson syringe, and into free air range from 10-40 lbf. The injection volume varies, typically ranging from 1.0 to 10.0 ml per injection, and multiple injection sites may be employed.

In other embodiments, the formulations of the present invention are injected via jet injection. Jet injection is a means of administering the dosage forms without the use of needles. Typically, a compression system (e.g., mechanical or gas) is used to accelerate the dosage forms to a relatively high velocity, allowing them to penetrate the tissue. Jet injector devices can be, for example, disposable, or reusable with medication cartridges that are prefilled or non-prefilled medication cartridges. Examples of jet injectors include Biojector® from Bioject, New Jersey, USA.

The invention is further described with reference to the following non-limiting Examples.

### EXAMPLES

### Example 1

Polyvinylpyrolidone (PVP) (K 90, BASF, Product # 09608802) is added to absolute, anhydrous ethanol (anhydrous 99.57%, Aldrich) while mixing in a beaker, wide mouth bottle or plastic jar using overhead stirrer with variable speed settings. The formulation is completed by stirring in calcium carbonate (CaCO₃)(EM Industries, Germany, catalog # EMCX0127-1). Formulation ranges are as follows:
PVP from 5% to 25 wt %
Ethanol 40% to 100 wt %
CaCO₃ 0.05% to 10 wt %
   Kinematic viscosity is measured using a Brookfield Kinematic Viscometer with CPE-40 cone spindle set at 0.5 rpm and 37° C. temperature, and found to be between 500 and 20000 cps.

### Example 2

5 wt % sodium alginate (FMC Biopolymer, Protonal LF 10/60) is dissolved in 30 grams D.I. water. Subsequently 7.5 grams of Sodium Chloride (NaCl) (VWR Scientific) are added, while mixing as described in Example 1, to form a gel. The formulation is completed by mixing in 1 wt % calcium carbonate (CaCO₃).

### Example 3

33,000 mg of salt is dissolved in 100 ml of DI water by mixing in a wide mouth glass or plastic jar. Subsequently, 3100 mg of CMC (Hercules Inc., Blanose Type 7HF PH, 9M31F PH or 7MF) polymer is quickly added into the salt solution to form a gel. The formulation is completed by mixing in 1 wt % calcium carbonate (CaCO₃). This particular formulation contains 1.30 wt % CMC, 13.84 wt % NaCl and 1 wt % calcium carbonate. General formulation ranges are as follows:
CMC from 1 wt % to 4 wt %
NaCl from 5 wt % to 30 wt %
CaCO₃ from 0.05 wt % to 10 wt %
   Kinematic viscosities for these formulations range from 29,000 to 36,000 cps.

### Example 4

About 3% by weight of hydroxypropyl cellulose (HPC) (Hercules Inc., Klucel Type HF or Type MF, Pharmaceutical Grade) is slowly added to absolute, anhydrous ethanol while stirring in a wide-mouth glass or plastic jar. The formulation is completed by mixing in 1 wt % calcium carbonate (CaCO₃). In general, formulation ranges are as follows:
HPC from 1% to 10 wt %
Ethanol 40% to 100 wt %
CaCO₃ 0.05% to 10 wt %
   Kinematic viscosity is typically between 36,000 and 42,000 centipoises for a formulation having about 3 wt % Klucel Type HF HPC. More generally, kinematic viscosity typically ranges from about 12,000 to 47,000 cps for HPC concentrations ranging from about 2 to 5 wt %.

### Example 5

NaCl is added to D.I. water, followed by CMC in sufficient quantities to yield a NaCl-CMC solution containing 330 mg/ml NaCl (or to saturation) and 40 mg/ml CMC. At the same time an alginate solution is prepared by adding sodium alginate to water at a concentration of 75 mg/ml. Equal volumes of the NaCl-CMC solution and the alginate solution are then mixed in a wide-mouth glass or plastic jar to form a viscous gel. The resulting gel contains 2% w/v CMC, 24% w/v NaCl, and 2.5% w/v alginate (which corresponds, if dry, to 7% w/w CMC, 84.21% w/w NaCl, and 8.77% w/w alginate), for a target kinematic viscosity between 32,000 and 36,000 cps.

More water can be added to decrease viscosity as desired. Additional formulations can be prepared by substituting additional ablation agents for the salt. Note that alginate is not soluble in high salt solution, hence the two step mixture. For other ablating agents besides salt, the mixture should be more straightforward (e.g., adding all the ingredients into the water).

The formulation is then injected into a prostate. Subsequently, the needle is retracted and a crosslinker (e.g., 2-20% w/w CaCl₂ in distilled water) is injected to crosslink part of the injected material, increasing the resistance of the injected gel to back-leakage.

### Example 6

The gel in Example 4 is injected into the prostate glands of 3 canines. For each dog, 0.4 cc of gel is injected (0.2 cc per side). After a period of 1 hour, the canine prostate (which is in the shape of a small walnut, ranging from 3.5 cc to 9.9 cc total volume) is harvested, sliced in 3 mm cross-sections, microtomed, and mounted on a slide for microscopic evaluation. The sections are labeled A, B, C, etc. and divided into Left and Right. A pathologist rates each section, and the results are presented in the table below.

For dog #I, the injection of 0.4 cc of gel resulted in the necrosis of 0.48 cc of tissue. For dog #II, the injection of 0.4 cc of gel resulted in the necrosis of 0.45 cc of tissue. For dog #III, the injection of 0.4 cc of gel resulted in the necrosis of 0.45 cc of tissue.

TABLE

| Dog # | I | | | | | | | | | | | | | | | | | |
|---|---|---|---|---|---|---|---|---|---|---|---|---|---|---|---|---|---|---|
| Section | A | A | B | B | C | C | D | D | E | E | F | F | G | G | H | H | | |
| | L | R | L | R | L | R | L | R | L | R | L | R | L | R | L | R | | |
| Necrosis | NP | NP | 3M | 0 | 4F | 3F | 3F | 3F | 2M | 2F | 2M | 2M | 2F | NP | 0 | | | |
| Marginal | | | 3 | 0 | 2 | 2 | 0 | 2 | 0 | 0 | 0 | 0 | 0 | 0 | | 0 | | |
| Vacuolization | | | | | | | | | | | | | | | | | | |
| Hemorrhage | | | | | | | | | | | | | | (4E) | | 0 | | |
| in necrosis | | | 2 | 0 | 3 | 3 | 3 | 2 | 2 | 2 | 2 | 2 | 1 | 0 | | | | |
| within glands | | | 0 | 0 | 2 | 0 | 0 | 1 | 1 | 1 | 2 | 1 | 0 | 1 | | | | |
| Inflammation | | | 0 | 0 | 0 | 0 | | 0 | | 0 | | | | | | 0 | | |
| acute | | | | | | | 2 | | 2 | | 1 | 2 | 2 | 0 | | | | |
| Dog # | II | | | | | | | | | | | | | | | | | |
| Section | A | A | B | B | C | C | D | D | E | E | F | F | G | G | H | H | I | I |
| | L | R | L | R | L | R | L | R | L | R | L | R | L | R | L | R | L | R |
| Necrosis | 0 | 0 | 0 | 0 | 0 | 0 | 0 | 2F | 2F | 3F | 3F | 4F | 3M | 3F | 2F | 2F | 0 | 1F |
| Marginal | 0 | 0 | 0 | 0 | 0 | 0 | 0 | 3 | 0 | 3 | 1 | 2 | 2 | 2 | 2 | 0 | 0 | 0 |
| Vacuolization | | | | | | | | | | | | | | | | | | |
| Hemorrhage | 0 | 0 | 0 | 0 | 0 | 0 | 0 | 0 | | | | | | | | | | |
| in necrosis | | | | | | | | | 1 | 3 | 2 | 2 | 2 | 2 | 1 | 2 | 0 | 2 |
| within glands | | | | | | | | | 0 | 0 | 0 | 0 | 0 | 0 | 2 | 0 | 2 | 1 |
| Inflammation | 0 | 0 | 0 | 0 | 0 | 0 | 0 | 0 | | 0 | 0 | | | | | | 0 | 0 |
| acute | | | | | | | | | 2 | | | 2 | 2 | 2 | 1 | 1 | | |
| chronic | | | | | | | | | | | | | | | 2 | | | |
| | | | | | | | | | | | | | | | (U) | | | |
| Cystic glands | | | | | | | | 2F | | | | | 1F | | | | | 2F |

| Dog # | III | | | | | | | | | | | | | | | | |
|---|---|---|---|---|---|---|---|---|---|---|---|---|---|---|---|---|---|
| Section | A | A | B | B | C | C | D | D | E | E | F | F | G | G | H | H | |
| | L | R | L | R | L | R | L | R | L | R | L | R | L | R | L | R | |
| Necrosis | NP | NP | 0 | 0 | 3F | 0 | 4M | 1F | 3F | 3F | 3F | 2F | 3F | 2F | 2F | 0 | |
| Marginal | | | | | 2 | | 2 | 0 | 0 | 0 | 2 | 2 | 1 | | 0 | 0 | |
| Vacuolization | | | | | | | | | | | | | | | | | |
| Hemorrhage | | | 0 | 0 | | 0 | | | | | | | | | | | |
| in necrosis | | | | | 2 | | 2 | 2 | 2 | 2 | 2 | 2 | 2 | 0 | 2 | 0 | |
| within glands | | | | | 0 | | 2 | | 2 | 0 | 2 | 3 | 3 | 2 | 3 | 2 | 3 |
| Inflammation | | | 0 | 0 | | 0 | | | | | | | | | | | |
| acute | | | | | 2 | | 2 | 2 | 2 | 2 | 3 | 2 | 3 | 2(3E) | 2 | 1(3E) | |
| chronic | | | | | 1FU | | | | | | | | | | | | |
| Cystic glands | | | 2M | 2M | | 2M | | 2M | 2F | 2M | 2M | 1M | 1M | 2M | 2F | | |

Key: 0 = finding not present; 1 p= minimal; 2 = mild; 3 = moderate; 4 = marked; 5 = severe; F = focal; M '2 multifocal; NP = no prostatic glands on section; L '2 left; R = right; E = extraprostatic; U = abnormally appears unrelated to foci of necrosis.

Although various embodiments are specifically illustrated and described herein, it will be appreciated that modifications and variations of the present invention are covered by the above teachings and are within the purview of the appended claims without departing from the spirit and intended scope of the invention.

1. An injectable formulation comprising: (a) a chemical ablation agent in an amount effective to cause tissue necrosis, and (b) a biodisintegrable viscosity adjusting agent in an amount effective to render the formulation highly viscous, wherein said injectable formulation is a sterile injectable formulation.

2. The injectable formulation of claim 1, wherein said ablation agent is an osmotic-stress-generating agent.

3. The injectable formulation of claim 1, wherein said ablation agent is an organic ablation agent.

4. The injectable formulation of claim 1, wherein said ablation agent is ethanol.

5. The injectable formulation of claim 1, wherein said ablation agent is a salt.

6. The injectable formulation of claim 1, wherein said ablation agent is sodium chloride.

7. The injectable formulation of claim 1, wherein said viscosity adjusting agent is present in an amount effective to provide a kinematic viscosity ranging from 5,000 cps to 100,000 cps.

8. The injectable formulation of claim 1, wherein said viscosity adjusting agent is present in an amount effective to provide a kinematic viscosity ranging from 10,000 cps to 50,000 cps.

9. The injectable formulation of claim 1, wherein said viscosity adjusting agent comprises a polysaccharide.

10. The injectable formulation of claim 9, wherein said viscosity adjusting agent is a polysaccharide selected from methylcellulose, hydroxyethylcellulose, hydroxypropyl cellulose, hydroxypropyl methyl cellulose, methylhydroxyethylcellulose, methylhydroxypropylcellulose, carboxymethyl cellulose and its salts, hydroxyethylcarboxymethylcellulose and its salts, carboxymethylhydroxyethylcellulose and its salts, alginic acid and its salts, hyaluronic acid and its salts, carageenan, chitosan, xanthan gum, guar gum, gum arabic, gum karaya, gum ghatti, konjac and gum tragacanth.

11. The injectable formulation of claim 1, wherein said viscosity adjusting agent comprises a polypeptide.

12. The injectable formulation of claim 11, wherein said viscosity adjusting agent is selected from gelatin and collagen.

13. The injectable formulation of claim 1, wherein said viscosity adjusting agent is selected from carboxyvinyl polymer, polyvinylpyrrolidone, polyacrylic acid, polyacrylamide, polyacilic acid/acrylamide copolymer, polyethylene oxide, polypropylene oxide, poly(ethylene oxide-propylene oxide), polymetaphosphate, polyethyleneamine, polypyrridine, as well as salts therof.

14. The injectable formulation of claim 1, further comprising an imaging contrast agent.

15. The injectable formulation of claim 14, wherein the imaging contrast agent is an MRI imaging contrast agent.

16. The injectable formulation of claim 14, wherein the imaging contrast agent is an ultrasonic imaging contrast agent.

17. The injectable formulation of claim 16, wherein the ultrasonic imaging contrast agent comprises a plurality of solid particles.

18. The injectable formulation of claim 17, wherein the plurality of solid particles is selected from calcium carbonate particles, hydroxyapatite particles, silica particles, poly(lactic acid) particles, and poly(glycolic acid) particles.

19. The injectable formulation of claim 1, wherein said injectable formulation comprises a plurality of viscosity adjusting agents.

20. The injectable formulation of claim 1, wherein said injectable formulation comprises a plurality of ablation agents.

21. The injectable formulation of claim 1, wherein said injectable formulation further comprises a liquid selected from water and an organic solvent.

22. A method of treatment comprising injecting the injectable formulation of any of claims 1-21 into the tissue of a subject.

23. The method of claim 22, wherein said tissue is prostatic tissue.

24. The method of claim 23, wherein said subject has been diagnosed with benign prostatic hypertrophy.

25. The method of claim 23, wherein the injectable formulation is transrectally injected into the subject.

26. The method of claim 23, wherein a plurality of injections are performed concurrently with a non-invasive imaging technique.

27. A prostatic ablation formulation comprising a prostatic ablation agent selected from free-radical generating ablation agents, oxidizing ablation agents and tissue fixing ablation agents.

28. The prostatic ablation formulation of claim 27, wherein the injectable prostatic formulation comprises a free-radical generating ablation agent.

29. The prostatic ablation formulation of claim 28, wherein the free-radical generating ablation agent is a peroxide compound.

30. The prostatic ablation formulation of claim 27, wherein the injectable prostatic formulation comprises an oxidizing ablation agent.

31. The prostatic ablation formulation of claim 27, wherein the injectable prostatic formulation comprises a tissue fixing ablation agent.

32. The prostatic ablation formulation of claim 31, wherein the tissue fixing ablation agent is selected from formaldehyde and glutaraldehyde.

33. A system for the chemical ablation of tissue, said system comprising:
(a) an injectable formulation comprising: (i) a chemical ablation agent in an amount effective to cause tissue necrosis, and (ii) a biodisintegrable viscosity adjusting agent in an amount effective to render the formulation highly viscous; and
(b) an apparatus for transcutaneously inserting said dosage form into said tissue.

34. The system of claim 33, wherein the apparatus is configured to insert said dosage form into the tissue transrectally.

35. The system of claim 33, wherein the tissue is prostatic tissue.

36. The method of claim 22, further comprising injecting a crosslinking agent into said tissue in an injection step separate from the injection of said injectable formulation.

37. The method of claim 36, wherein said crosslinking agent is injected subsequent to the injection of said injectable formulation.

38. The injectable formulation of claim 1, wherein said injectable formulation comprises an ionically crosslinkable polymer.

39. The injectable formulation of claim 38, wherein said ionically crosslinkable polymer is an alginate polymer.
